(19) United States
(12) Patent Application Publication
(10) Pub. No.: US 2010/0022633 A1
(43) Pub. Date: Jan. 28, 2010

(54) Cognitive abilities improving agent

(76) Inventors: Mathias Alfons Everhard Frevel, Waihi Beach (NZ); Andrew Pipingas, Victoria (AU); Richard Silberstein, Victoria (AU); Larry Ellsworth Stenswick, Rangiora (NZ)

Correspondence Address: DUANE MORRIS LLP - San Diego, 101 WEST BROADWAY, SUITE 900, SAN DIEGO, CA, 92101-8285, US

(21) Appl. No.: 11/658,965

(22) PCT Filed: May 22, 2006
(86) PCT No.: PCT/NZ06/00122

§ 371 Date: Sep. 28, 2009

(30) Foreign Application Priority Data
May 23, 2005 (NZ) 540257

(51) Int. Cl. A61K 31/352; A61P 25/28

(52) U.S. Cl. 514/456

(57) ABSTRACT

A composition containing bark extract from the genus Pinus is described to improve, or prevent a decline in, human cognitive abilities or improve, or prevent symptoms of, neurological disorders in a human. The composition is advantageous for treatment where the human does not suffer from any kind of neurodegenerative disease at administration; the decline in cognitive abilities is age-associated, and is not associated with any neurodegenerative disease; or where the neurological disorders are associated with symptoms characterised by a loss in cognitive abilities.

### TECHNICAL FIELD

The invention relates to a cognitive abilities improving agent. More specifically, the invention relates to bark extract compositions and new uses and methods of treatment to improve or, prevent decline in cognitive abilities such as memory function.

### BACKGROUND ART

The human brain is a very complex system with a multitude of cognitive functions. Physiologically the brain can be divided into several parts that are involved to different extents in processing information relevant to the performance of different cognitive tasks. On a neurophysiological level, excitatory and inhibitory populations of neurons are involved that build neural networks of regions, sub-regions and interregional connections. On an electrophysiological level, there are excitatory and inhibitory electrical potentials involved within and across this network. Together, the complex functioning of this network is understood to be the basis for complex cognitive abilities that enable us to learn, comprehend, reason and remember.

An important part of brain processing that is involved in most human cognitive abilities is working memory, which comprises and goes beyond the older concept of short-term memory. Working memory refers to the brain's ability to provide temporary storage and manipulation of the information necessary for such complex tasks as language comprehension, learning and reasoning. The working memory is currently thought of as a system of several subcomponents with a central executive function that coordinates these different resources. The central executive function is important in skills like chess playing and is particularly susceptible to the effects of Alzheimer's disease (Baddeley, 1992).

Individual human beings differ in their cognitive abilities. For example, people have different abilities to memorise words, numbers or pictures and differ in the reaction speed and accuracy when asked to recall information, or perform intellectually demanding tasks like playing chess.

Cognitive performance of an individual can be tested and assessed with many different types of cognitive tasks. In conjunction with sophisticated neurophysiological and neuro-imaging techniques one can detect different brain activities in and between different brain regions in persons performing cognitive tasks. This approach makes it possible to define certain correlations between cognitive performance on different tasks and characteristic brain activities.

Examples of such correlations include:
That working memory capacity correlates very highly with an individuals' reasoning skills assessed by standardised intelligence tests (Colom, 2004);
Or that individuals of higher intelligence (IQ) have more negative electrical brain potentials when performing a certain task compared to lower intelligence (IQ) individuals (Pelosi L et al, 1992).

This type of testing allows measurement of cognitive performance, and to determine improvement or decline in cognitive abilities. Studies have shown that cognitive functions, including memory abilities, decline with normal aging and, as a consequence of neurological diseases; the extent of change in the different types of cognitive function varies (Cullum et al, 2000).

In one study, cognitive abilities including short term memory and working memory were shown to be compromised with age, as demonstrated by significant decreases in performance on a computerised test of spatial working memory with increasing age of the populations tested (Tournier et al, 2004).

Very limited treatment options exist to:
Improve cognitive abilities including memory in healthy people of any age;
To prevent the normal decline in cognitive abilities with age;
To counteract the symptoms of cognitive impairment characteristic of many neurological diseases including dementia in people.

A traditional treatment that has been investigated in numerous clinical studies is the use of dietary supplementation with ginkgo biloba extracts alone and in combination with other plant extracts. Scientific studies on the efficacy of ginkgo biloba have given very inconsistent results in numerous publications with some finding no effect and others showing memory improvements.

PCT publication WO 2002/053152 considers use of any flavonoid including proanthocyanidins from flavonoid sources such as grape seed or green tea to treat brain cancers and neurodegenerative diseases specifically Parkinson's disease. The usefulness of flavonoids for this purpose of treating neuro-degenerative diseases is inferred by the teachings that 1) neuro-degeneration can result from increased oxidative stress in form of free radical damage to cells, that 2) Parkinson's patients show signs of such damage, and that 3) flavonoids can act as antioxidants to counteract oxidative stress. However, for someone skilled in the art it is obvious that these teachings only constitute concurrent observations, have no causal relationships, and do not reasonably infer the expectation that any flavonoid or flavonoid combination could be used to treat neurodegenerative diseases. The specific example provided in WO 2002/053152 refers to the effects of tangeretin, a specific flavonoid from the peel of citrus fruit, in a chemically induced rat brain model of Parkinson's disease. From the teachings in this example a person skilled in the art can not expect that any flavonoid other than tangeretin may potentially have a positive effect in Parkinson's disease if at all. The applications described in this PCT publication are limited to treatment of brain diseases that exhibit a physical manifestation such as brain cancer and Parkinson's disease. No disclosure is made regarding memory or improving/preventing decline in memory.

US patent application no. 2003/0180406 describes a method using polyphenol compositions specifically derived from cocoa to improve cognitive function. The mechanisms however are related to gap junctional communications between cells and more specifically to abundance of connexin proteins in cell membranes. Evidence for the claimed effects is restricted to gap junctional cell to cell communication, and is only from cell culture experiments on rat liver epithelial cells. No effects on cognitive function or neurodegenerative diseases in humans are demonstrated or could reasonably be expected by someone skilled in the art from the examples shown in US application no. 2003/0180406. No effects on human brain activities are described that may correspond to improved cognitive functions, such as memory.

A recent pharmacological treatment option of Alzheimer's dementia is the use of acetylcholinesterase inhibitors. These compounds can slow down progression but not cure the disease and can exert unfavourable cholinergic, hepato-toxic and other side-effects. Pharmacological enzyme inhibitors are also unsuitable for use in healthy people to enhance cognitive abilities due to risk of undesirable side-effects.

Patent publication WO 2005/044291 describes use of grape seed (Vitus genus) extract to protect neuronal cells and to therefore prevent degenerative brain diseases. Brain diseases are described as including stroke, cerebral concussion, Huntingtons disease, CJD, Alzheimer's, Parkinsons, and senile dementia. No teaching is provided for use of other extracts to provide similar functions nor would it be obvious to expect the same results from other extracts owing to the unique flavonoid profile of grape seed extract. Further, the invention is not directed to non-disease states such as elective treatments to improve cognitive abilities.

Patent publication WO 2005/067915 discloses a synergistic combination of flavonols and hydroxystilbenes (synthetic or from green tea) combined with flavones, flavonols, proanthocyanidins and anthocyanidins (synthetic or from bark extract). The combination is described as being used to reduce neuronal degeneration associated with disease states such as dementia, Alzheimer's, cerebrovascular disease, age-related cognitive impairment and depression. The individual components are not disclosed as having a therapeutic effect in their own right. In addition, the combination is used to treat humans who are already in a disease state. Treating humans who are free from neuronal disease as an elective treatment is not disclosed nor would it be obvious form the description.

U.S. Pat. No. 5,719,178 describes use of proanthocyanidin extract to treat ADHD. The patent describes that ADHD may be equivalent to a persistent pattern of inattention and/or hyperactivity impulsivity and is termed a cognitive deficit. The proanthocyanidin extract used in patent U.S. Pat. No. 5,719,178 is extracted from conifer (pine) bark (maritime pine) and is used in conjunction with heterocyclic anti-depressant (desipramine) and a citrus bioflavonoid. As with the above patent publications, the individual components (proanthocyanidin, anti-depressant and flavonoid) are not disclosed as having any useful therapeutic effect in their own right. In addition, the combination is used to treat humans who are already in a disease state (have ADHD). Treating humans who are free from neuronal disease as an elective treatment to improve general cognitive ability is not disclosed nor would it be obvious form the description.

Patent publication WO 02/076381 describes methods of treating amyloid, non-amyloid component and α-synuclein diseases in mammals by administration of proanthocyanidins. Proanthocyanidins are described as being-potent inhibitors of amyloid and α-synuclein/NAC fibrillogenesis and cause potent disruption/disassembly or pre-formed fibrils for a variety of amyloids and α-synuclein diseases associated with Alzheimer's, Parkinson's, type 2 diabetes, systemic AA amyloidosis and other amyloid diseases. It is unclear, however, how the presence of amyloid or a-synuclein fibrils or the process of fibrillogenesis in the multiple amyloid diseases is related to loss of cognitive function and whether there is any cause effect relationship. Furthermore, treating humans who are free from neuronal disease as an elective treatment to improve general cognitive ability is not disclosed nor would it be obvious from the description.

Accordingly, it should therefore be appreciated that it would be desirable to have a formulation for use in improving cognitive function and/or preventing decline in cognitive function that does not include unfavourable side-effects that may be associated with existing treatments.

It is therefore an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

It has been found by the applicant that by taking an oral dose of compositions containing a bark extract, human subjects perform better on tasks designed to test cognitive abilities.

For the purpose of this specification the term ‘cognitive abilities’ is defined as the result of the mental processes taking place in the brain that are involved in information processing, including: intelligence, learning ability, problem solving ability, the ability to concentrate, memory, comprehension, executive functions, working memory, response times on cognitive tasks, accuracy on cognitive tasks.

The terms ‘improve’, ‘improving’ or ‘improvement’ or grammatical variations thereof used in relation to cognitive functions refer to the ability to achieve a measurable increase in performance in relation to tasks used to test these cognitive functions in humans.

The term ‘memory’ is defined as the biological processes of the brain that enable storage and recall of information.

The term ‘working memory’ is defined as a combination of processes of the brain that provide temporary storage and manipulation of information necessary to perform complex cognitive tasks such as learning and reasoning.

The term ‘concentrate’ or grammatical variations thereof refers to the ability of a subject to focus on a particular task without being distracted.

The term ‘accuracy’ or grammatical variations thereof refers to the ability to make correct decisions on tasks used to test these cognitive functions in humans.

The terms ‘higher intelligence’ and ‘lower intelligence’ refer to relative comparisons of individuals on the basis of standard psychological intelligence quotient (IQ) tests. For example, a person would be considered to be of higher intelligence when scoring a value of 120 in a full-scale IQ test compared to a person of lower intelligence that scored a value of 100 on the same test.

The term ‘response time’ refers to the time it takes for a subject to respond to a task that requires a decision to be made. The response time is measured from when the subject encounters a stimuli of some form, for example a question, to when the subject reacts to the stimuli, for example by giving an answer or pressing a ‘yes/no’ button.

The term ‘effective amount’ or ‘dose’ or grammatical variations thereof refers to an amount of agent sufficient to exhibit the desired effects. The effective amount may be determined by a person skilled in the art using the guidance provided herein.

According to one aspect of the present invention there is provided a composition to improve, or prevent a decline in, human cognitive abilities,

wherein the human does not suffer from any kind of neurodegenerative disease at administration and,

wherein the composition includes a therapeutically effective amount of bark extract from the genus Pinus.

According to a further aspect of the present invention there is provided a composition to improve, or prevent a decline in, cognitive abilities in humans,
wherein the decline in cognitive abilities is age-associated, and is not associated with any neurodegenerative disease and,
wherein the composition includes a therapeutically effective amount of bark extract from the genus Pinus.

According to a further aspect of the present invention there is provided a composition including a pharmacological agent to improve, or prevent symptoms of, neurological disorders in a human,
wherein the symptoms are characterised by a loss in cognitive abilities, and wherein the pharmacological agent includes a therapeutically effective amount of bark extract from the genus Pinus.

According to a further aspect of the present invention, there is provided the use of a therapeutically effective amount of an extract obtained from the bark of trees of the genus Pinus in the manufacture of a composition,
to improve, or prevent a decline in, cognitive abilities in a human,
wherein the human does not suffer from any neurodegenerative disease when the composition is administered to the human.

According to a further aspect of the present invention, there is provided the use of a therapeutically effective amount of an extract obtained from the bark of trees of the genus Pinus in the manufacture of a composition,
to improve, or prevent a decline in, cognitive abilities in humans,
wherein the decline in cognitive abilities is age-associated, and is not associated with any neurodegenerative disease at the time of administration to the human.

According to a further aspect of the present invention there is provided the use of a therapeutically effective amount of an extract obtained from the bark of trees of the genus Pinus in the manufacture of a composition,
to improve, or prevent symptoms of neurological disorders in a human,
wherein the symptoms are characterised by a loss in cognitive abilities.

According to a further aspect of the present invention there is provided a method of improving, or preventing a decline in, human cognitive abilities,
wherein the human is administered a composition containing a therapeutically effective amount of an extract obtained from the bark of trees of the genus Pinus and,
wherein the human does not suffer from any neurodegenerative disease when the composition is administered.

According to a further aspect of the present invention there is provided a method of improving, or preventing a decline in, cognitive abilities in humans,
wherein the human is administered a composition containing a therapeutically effective amount of an extract obtained from the bark of trees of the genus Pinus and,
wherein the decline in cognitive abilities is age-associated, and is not associated with any neurodegenerative disease at the time of administration.

According to a further aspect of the present invention there is provided a method of improving, or preventing symptoms of neurological disorders in a human,
wherein the human is administered a composition containing a therapeutically effective amount of an extract obtained from the bark of trees of the genus Pinus and,
wherein the symptoms are characterised by a loss in cognitive abilities.

Preferably, the cognitive abilities are selected from one or more of: intelligence, learning ability, problem solving ability, the ability to concentrate, memory, comprehension, executive functions, working memory, response times on cognitive tasks, accuracy on cognitive tasks.

Preferably, the active compounds in the composition are water soluble and extracted using a water based extraction process. One example process is that of NZ 329658/U.S. Pat. No. 5,968,517.

Preferably, the composition includes a complex mixture of flavonoids with some non-flavonoid compounds. Most preferably, the bark extract is sourced from the bark of Pinus radiata (the Monterey pine or radiata pine). Pine bark extract has been found by previous research to be non-toxic to mice and humans, and have a broad spectrum of actions that are beneficial to human health.

Based on previous research it is the inventor's understanding that the preferred composition is rich in a variety of proanthocyanidin compounds that are dimers, trimers, oligomers and polymers of catechin, and epicatechin, and also contains one or more other flavonoids and plant-phenolic compounds that may be selected from the group including: catechin, epichatechin, gallocatechin, epigallocatechin, quercetin, dihydroquercetin, myricetin, astringenin, pinosylvin, taxifolin, stilbenes, hydroxylstilbenes, and phenolic acids.

Preferably, the composition is produced using a water-based extraction process.

In preferred embodiments, the composition is administered orally. Alternatively, other methods of administration are also envisaged such as: suspensions, drinks, tonics, syrups, powders, ingredients in solid or liquid foods, and combinations thereof.

In preferred embodiments, the composition substantially as described above may be administered orally as a regular daily dose.

It is the inventor's experience that a preferred dose may be a total of 1 to 10 capsules taken orally per day (more preferably, 4 capsules per day), with each capsule containing approximately 240 mg of pine bark extract or proanthocyanidins per day. It should be appreciated that dose rates may vary between these levels depending on the metabolism level of the subject and other biochemical factors, such as seasonal dietary requirements, method of administration, extract potency and the like.

It should also be appreciated by those skilled in the art that a dosage lesser or greater than that above may also be used without harm and may still be effective. Pine bark extract is non-toxic, has a naturally occurring source, and is a proanthocyanidin-rich substance. Higher doses of at least this extract would not produce any toxic reactions to the subject and may in fact be advantageous for some subjects. Lower doses may also be advantageous and achieve the same efficacy in treatment.

The treatments/preventative administration may be continued as a regimen, i.e. for an effective amount of time, e.g. daily, weekly, monthly, bimonthly, biannually, annually or in some other regime as determined by a person skilled in the art for such time as necessary. The administration may be continued for at least a period of time sufficient to improve cognitive abilities. Preferably, the regimen is continued on a daily basis for at least 5 weeks.

In one embodiment, it is understood by the inventors that the bark extract may alter brain electrical activities and through this alteration the new pattern of brain electrical activities may resemble those of persons with higher intelligence compared to those with lower intelligence as determined by standard psychological tests. Whilst this is one understanding, it should be appreciated that the neurological system is very complex and as such, other mechanisms not contemplated by the inventors may also be occurring.

It is also an understanding that the bark extract may improve, or prevent decline in, patterns brain electrical activities. Preferably, the bark extract may improve or prevent the decline of brain physiological processes. Preferably, these are processes that lead to the impairment of cognitive functions. Again, the above understanding should not be seen as limiting.

The main advantages found are that the subject performs better on memory tasks compared to subjects who are not treated, and that brain electrical activities are altered in a favourable manner.

A further advantage of the present invention is that it appears to have no adverse side effects after treatment with the composition of the present invention. This is a major improvement on the prior art where adverse side effects are a common occurrence for pharmaceutical drugs that aim to counteract loss of cognition in humans with neurological diseases.

In one embodiment, the composition may also include other antioxidant active components including: flavonoids, vitamins, minerals, other known therapeutically active compounds, additives or excipients. However, it should be appreciated by those skilled in the art that the composition may be effective even it only includes compounds extracted from pine bark.

In a further option the composition, substantially as described above, may also be formulated using components selected from: fillers, excipients, modifiers; humectants, stabilisers, emulsifiers, and other known formulation components.

It should be appreciated from the above description that there is provided a composition and associated uses and methods for treating a range of cognitive functions, particularly those surrounding memory. The methods may be used even in healthy subjects to improve cognitive function or at least prevent decline in cognitive function. No adverse side effects are noted from the composition hence the methods may provide a useful alternative to existing methods and treatments.

### BRIEF DESCRIPTION OF DRAWINGS

Further aspects of the present invention will become apparent from the following description which is given by way of example only and with reference to the accompanying drawings in which:

FIG. 1 Shows an illustration of the distribution of electrode pairs where the difference between start of the trial and completion exceeds the critical significance value of t>2.86 for the pine bark extract group in an object working memory task;

FIG. 2 Shows an illustration of the distribution of electrode pairs where event related partial coherence (ERPC) was correlated with individual accuracy in an object working memory task;

FIG. 3 Shows an illustration of the distribution of electrode pairs where the event related partial coherence (ERPC) difference exceeds t=2.86 in a paired Student t-test for a pine bark extract group; and,

FIG. 4 Shows an illustration of the distribution of electrode pairs where the event related partial coherence (ERPC) for correct trials minus the ERPC for incorrect trials before supplementation exceeds the critical value of t>2.72.

### BEST MODES FOR CARRYING OUT THE INVENTION

The invention methods and uses are now described with reference to a double-blind, controlled clinical trial with subjects in treatment and control groups to determine the effects from administration of a pine bark extract on cognitive function in humans.

### Methodology

### Supplement

A pine bark extract from the bark of Pinus radiata was used in the trial treatment group in the form of capsules to be taken orally. Each capsule contained a dose of approximately 240 mg pine bark extract and approximately 30 mg vitamin C. In the trial control group placebo capsules of equal appearance but only containing 30 mg of vitamin C were used.

### Subjects

The trial was completed on a total of 42 people all aged between 50 and 65 years. Participants were enrolled into the study on the basis that they were in good general health and through confirmation that they fulfilled all inclusion and exclusion criteria as determined by a general practitioner. Criteria included not taking any form of vitamin or herbal supplementation and not suffering from any neurological disorder prior to the trial.

Subjects were randomly assigned to receive either pine bark extract or the placebo supplement for the duration of the trial period (5 weeks). Participants did not take any other supplements during the trial. Both the participant and the research assistant involved in analysing the results were blinded to the type of supplementation (pine bark extract or placebo).

Participants were required to take a daily dose of 4 capsules. Those assigned to pine bark extract supplementation received a total daily dose of 960 mg of pine bark extract and 120 mg of vitamin C from the 4 capsules. Participants assigned to placebo supplementation were required to take 4 capsules daily containing in total 120 mg of vitamin C. The placebo capsules were identical in appearance to the pine bark extract containing capsules.

### Testing

Brain electrical neuro-imaging and cognitive tests were completed before supplementation began (session 1) and again after 5 weeks of supplementation (session 2).

Computerised cognitive testing and steady-state probe topography (SSPT) neuro-imaging was used to detect rapid changes in brain activity and changes in memory performance measures. The electrophysiological technique of SSPT is used here to investigate changes in brain electrical activity associated with dietary supplementation because brain electrical activity is instantaneous and is sensitive to the level of neurotransmitters, and can more easily be measured using this technique. With the SSPT technique, neuro-imaging memory processes that occur with sub-second timing can be detected as changes in electrical signals over specific brain regions. These patterns of activity can be averaged over successive stimulus presentations to produce reliable estimates of the overall changes in electrical activities for individual subjects during the performance of cognitive tasks. More information regarding SSPT neuro-imaging has been published elsewhere (Silberstein et al., 1990; Silberstein et al., 1995).

During sessions 1 and 2, before and after 5 weeks of supplementation, brain electrical activity was recorded continuously at 64 scalp sites while subjects performed a series of screen based cognitive tasks. Superimposed on the viewing field screen was a continuously presented light flicker delivered by way of a set of goggles with half mirrored lenses subtending horizontal and vertical angles of 160° and 90° respectively. This flicker is termed the ‘irrelevant’ or ‘probe flicker’ because subjects concentrate on the computer-based tasks and not on the flicker, though it is the flicker that generates the steady-state visually evoked potential (SSVEP).

During computerised cognitive testing, accuracy and response time were measured for each participant in session 1 and session 2 for each cognitive task. The information was analysed using mixed design ANOVA to investigate possible interactions of group (pine bark extract, placebo) and time (session 1, session 2) and repeated measures ANOVA used to investigate time effects for each group separately.

During brain electrical neuro-imaging, Steady-State Visually Evoked Potential Event Related Partial Coherence (SSVEP-ERPC) was measured for all 2016 distinct pairs of electrodes on the scalp and averaged across all trials. The ERPC provides a measure of functional connections between cortical regions.

The relationship between psychometric measures such as accuracy and SSVEP-ERPC was also measured for specific electrode pairs by calculating the correlation coefficient between these measures for each time point in the task epoch repeated for all 2016 electrode pairs. After several levels of significance testing, the partial coherence measure and psychometric performance was compared between results gathered in session 1 and session 2 to identify differences in brain activities between the pine bark extract group and the placebo group.

### Results

### Special Working Memory and Simple Recognition Memory

Analyses of the results from the computerised cognitive testing showed a significant improvement in response times for special working memory and simple recognition memory in the pine bark extract group but not in the placebo group. Table 1 shows results for neuro-cognitive response time scores (milliseconds, ms) from Individual neuro-psychological tests at pre-treatment and post-treatment for participants administered pine bark extract or placebo capsules.

TABLE 1

| | Response Time (ms) | | | | | | | |
|---|---|---|---|---|---|---|---|---|
| | Participants administered | | | | Participants administered | | | |
| | pine bark extract (n = 22) | | | | placebo (n = 20) | | | |
| | Pre-treatment | | Post-treatment | | Pre-treatment | | Post-treatment | |
| Cognitive Task | Mean | SD | Mean | SD | Mean | SD | Mean | SD |
| Spatial Working | 1017.7 | 145.1 | 953.3 | 111.2 | 946.1 | 132.7 | 947.6 | 166.8 |
| Memory | | | | | | | | |
| Simple Recognition | 1106.6 | 113.3 | 1046.9 | 105.6 | 1049.9 | 134.0 | 1056.9 | 141.5 |
| Memory | | | | | | | | |

For the pine bark extract group, the mean response time for spatial working memory improved by 64 ms after supplementation (time effect significance p=0.04). There was a non-significant increase in reaction time by 1.5 ms in the placebo group. This change in the pine bark extract group was significant over placebo by univariate analysis (p<0.05).

For the pine bark extract group, the mean response time for simple recognition memory improved by 59.7 ms after supplementation (time effect significance p=0.0016). There was a non-significant increase in reaction time by 7 ms in the placebo group. This change in the pine bark extract group was significant over placebo (p<0.05) by univariate analysis.

These results show significant improvements in important memory components in the pine bark extract group that where not observed in the placebo group.

### Brain Electrical Neuro-Imaging

Analyses of the results from brain electrical neuro-imaging showed surprising and unexpected effects for participants taking pine bark extract supplementation that were not present in the placebo group.

Event related partial coherence (ERPC) analyses of brain electrical activity measurements were taken during an object working memory task and a retrieval memory task.

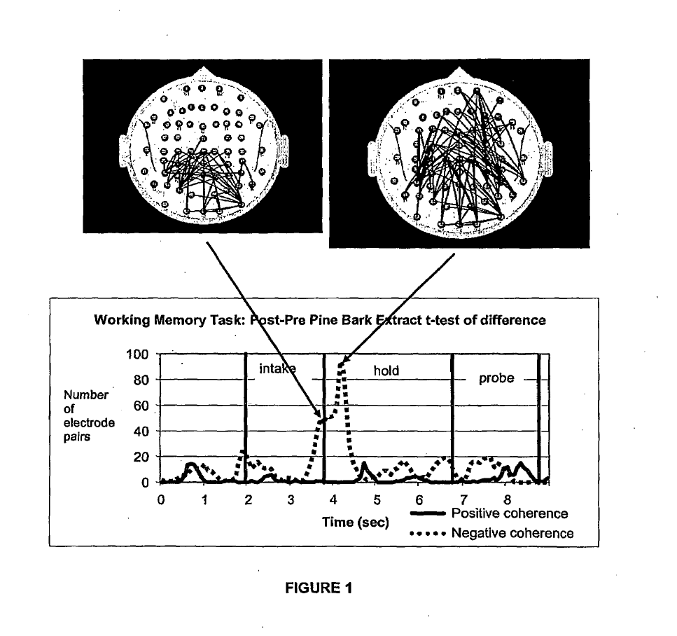

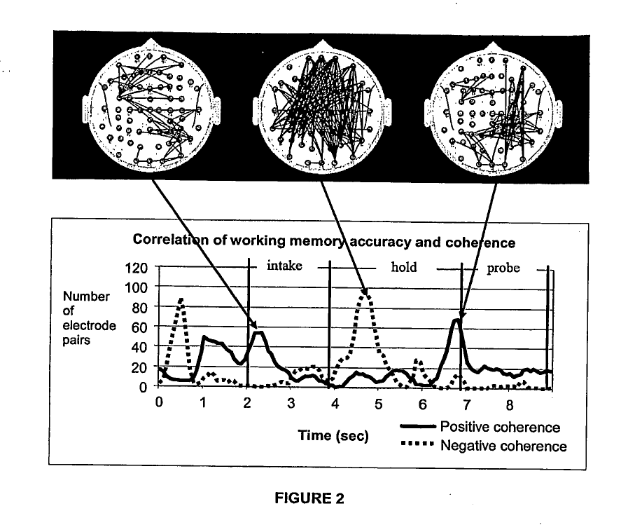

The results for the object working memory task (FIGS. 1 and 2) showed that there was a change in coherence patterns for the pine bark extract group after supplementation that indicated long-range decoupling of frontal/pre-frontal and parieto-temporal sites of the brain in the hold period of the memory task. FIG. 1 illustrates the distribution of electrode pairs where the session 2 and session 1 difference exceeds the critical significance value of t>2.86 for the pine bark extract group. Note that the peak effect for the pine bark extract group occurs at the beginning of the hold interval in the object working memory task and primarily comprises long-range decoupling. As shown in FIG. 2, it was found using correlation analyses of the distribution and nature of ERPC with individual performance level, that the accuracy of response correlated very significantly with long-range decoupling frontal/pre-frontal and parieto-temporal sites during this hold period of the memory task. Note that the dotted line in FIG. 2 corresponds to electrode pairs where ERPC is positively correlated with accuracy. The peak of highest significance occurs in the hold phase and shows long-range decoupling.

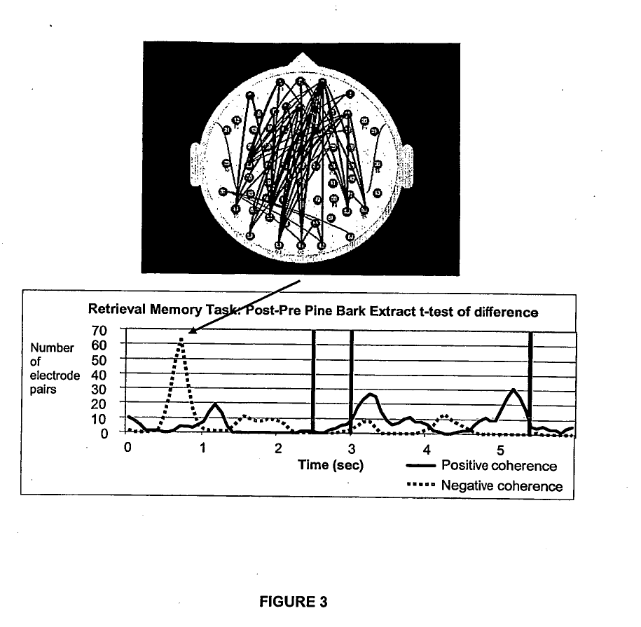

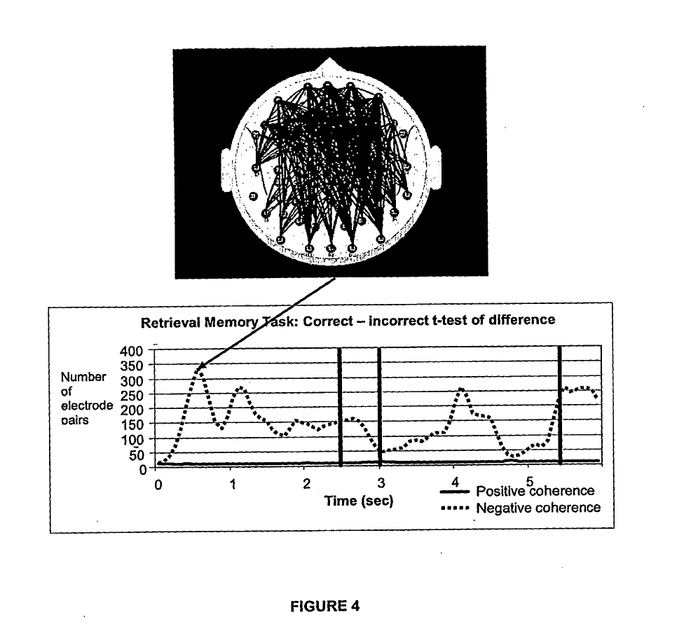

The results of ERPC analyses of the retrieval memory task (FIGS. 3 and 4) also showed that there was a change in coherence patterns for the pine bark extract group after supplementation that indicated long-range decoupling of frontal/pre-frontal and parieto-temporal sites of the brain approximately 0.6 seconds after presenting of the probe. FIG. 3 shows an illustration of the distribution of electrode pairs where the ERPC difference exceeds t=2.86 in a paired Student t-test for the pine bark extract group with a highly significant peak of negative coherence at around 0.6 seconds. The significance of the long-range decoupling observed in this memory task was examined in terms of enhanced cognition or otherwise by measuring the difference between correct and incorrect retrieval trials.

FIG. 4 shows an illustration of the distribution of electrode pairs where the ERPC for correct trials minus the ERPC for incorrect trials in session 1 exceeds the critical value of t>2.72. The graph shows the number of electrode pairs where the Student t-test for the correct-incorrect comparison exceeded the critical value of |t|>2.72. The comparison of the ERPC for correct minus incorrect trials indicated that correct trials were associated with reduced cortical coupling, especially around 0.6 seconds after the presentation of the probe as depicted by the highest peak in the dotted line.

Described another way, the above findings indicate that reduced cortical coupling is linked to correct performance on the above memory tasks indicating transiently enhanced cognition during task performance. Importantly, this observed reduction in long-range cortical coupling occurred only in the pine bark extract group but not the placebo group when they performed the memory tasks. This indicates that those in the pine bark extract group exhibited changes in brain electrical activities that manifested in enhanced cognitive processes when completing memory tasks.

Furthermore, other studies that have investigated brain electrical activities in people of different IQ levels have shown that:
1. ‘More negative’ event related potentials when performing a cognitive task correlate strongly with higher intelligence levels (Pelosi L et al 1992); and,
2. Increased individual proficiency correlates with reduced long-range cortical coupling between prefrontal/frontal sites and posterior sites on a mental rotation trial (Silberstein, personal communication).

These observations support the conclusion that the changes in brain activity observed in the pine bark extract group simulate brain activity patterns that resemble those found in people of higher IQ.

### Trial Conclusions

The analyses showed that:
Pine bark extract supplementation improved speed of response in people performing memory tasks.
There was a pattern of brain electrical activity specific to the pine bark extract group;
This pattern correlated with increased accuracy of response in the object memory task and correct trial performance in the retrieval memory task;
Hence, indicated enhanced cognitive processes in this group.
The particular type of brain activity pattern was a long-range decoupling of brain regions in certain phases of the memory task;
These particular patterns of brain activity indicated a more efficient enhanced brain capacity that occurred to prevent unnecessary brain activities distracting from the task that the subject was concentrating on.

By inference, subjects may also have increased intelligence since similar patterns of inhibitory brain electrical activities have been found to correlate with IQ levels and either prevention and/or treatment of neurological disorders or symptoms thereof.

### Trial Summary

Pine bark extract improved speed of response on a spatial working memory task and a simple recognition memory task, whereas there was no change with placebo supplementation. Pine bark extract was also found to exhibit significant changes in brain cortical coupling between sessions 1 and 2 during performance of working memory and recognition memory tasks.

The findings suggest that the neural networks essential for encoding information in working or longer term memory operate more effectively as a consequence of pine bark extract treatment. The findings further indicate that pine bark extract treatment modifies cortical function in a manner that enhances cognitive processes, in particular both working and recognition memory.

It should be appreciated that the improvements noted above show that pine bark extract supplementation may be a useful treatment for improving, or at least preventing decline in, cognitive abilities and, given the similar brain patterns noted as people of high IQ it may be inferred that pine bark extract also improves, or prevents decline in, intelligence.

It should further be appreciated that the improvements in memory performance and in brain electrical activity suggest pine bark extract supplementation may also provide a method of preventing or treating normal age-related decline in memory, or neurological disorders that are characterised by decline in cognitive functions such as different types of dementia.

It should further be appreciated that the changes in brain electrical activity that indicated decoupling or an increase in inhibitory processes suggest pine bark extract supplementation may also provide a method of preventing or treating disorders or symptoms thereof that share abnormal brain electrical hyperactivities as seen in cases of epilepsy or depression.

As the treatment is unlikely to exhibit any side effects, it has a significant advantage over existing neurological treatments in that it may be used equally for both healthy people and those people with cognitive dysfunctions.

Aspects of the present invention have been described by way of example only and it should be appreciated that modifications and additions may be made thereto without departing from the scope thereof as defined in the appended claims.

### REFERENCES

Baddeley, A. ‘Working memory’. Science 255: 556-559; 1992.
Colom, R; Rebollo, I; Palacios, A; Juan-Espinosa, M; Kyllonen, P. C. ‘Working memory is (almost) perfectly predicted by g’. Intelligence 32: 277-296; 2004.
Cullum, S; Huppert, F A; Mcgee, M; Dening, T; Ahmed, A; Paykel, E S, et al. ‘Decline across different domains of cognitive function in normal ageing: results of a longitudinal population-based study using CAMCOG’. International Journal of Geriatric Psychiatry 15: 853-862; 2000.
Pelosi, L; Holly, M; Slade, T; Hayward, M; Barrett, G; Blumhardt, L D ‘Event-related potential (ERP) correlates of performance of intelligence tests’. Electroencephalography and Clinical Neurophysiology/Evoked Potentials Section 84: 515-520; 1992.
Tournier, E; Pipingas, A; Stough, C K (2004) ‘Detecting age-associated cognitive decline: effects on episodic memory performance’. Presented at the 14th Australasian Society for Psychophysiology Conference. December 2004.
Silberstein, R B; Schier, M A; Pipingas, A; Ciorciari, J; Wood, S R; Simpson, D G (1990) ‘Steady-state visually evoked potential topography associated with a visual vigilance task’. Brain Topography, 3, 337-347.
Silberstein, R B; Ciorciari, J; Pipingas, A (1995) ‘Steady-state visually evoked potential topography during the Wisconsin card sorting test’. Electroencephalography and Clinical Neurophysiology, 96, 24-35.

1-24. (canceled)

25. A method of improving cognitive ability in a human, comprising administration of a composition to said human which comprises an extract obtained from the bark of trees of the genus Pinus in an amount effective to improve cognitive abilities in said human, wherein the human does not suffer from a neurodegenerative, neurological, psychiatric or behavioral disorder at the time of administration.

26. A method according to claim 25, wherein the cognitive ability is memory.

27. A method according to claim 25, wherein the cognitive ability is working memory.

28. A method according to claim 25, wherein the cognitive ability is the ability to concentrate.

29. A method according to claim 25, wherein the cognitive ability is cognitive processing speed.

30. A method according to claim 25, wherein the cognitive ability is one or more of learning ability, problem solving ability, comprehension, response times on cognitive tasks, and accuracy on cognitive tasks.

31. A method according to claim 25, wherein the extract is from the bark of the Pinus radiata.

32. A method according to claim 25, wherein the extract includes a mixture of flavonoid and non-flavonoid compounds.

33. A method according to claim 25, wherein the extract comprises proanthocyanidin compounds.

34. A method according to claim 33, wherein said proanthocyanidin compounds comprise dimers, trimers, oligomers and polymers of catechin and epicatechin.

35. A method according to claim 33, wherein said extract further comprises one or more other flavonoids and plant-phenolic compounds selected from the group consisting of catechin, epichatechin, gallocatechin, epigallocatechin, quercetin, dihydroquercetin, myricetin, astringenin, pinosylvin, taxifolin, stilbenes, hydroxylstilbenes, and phenolic acids.

36. A method according to claim 25, wherein the extract contains water soluble active compounds.

37. A method according to claim 25, wherein the extract is produced using a water-based extraction process.

38. A method according to claim 25, wherein the extract is administered daily.

39. A method according to claim 31, wherein the extract is administered daily.

40. A method according to claim 25, wherein the extract is administered daily for at least about five weeks.

41. A method according to claim 31, wherein the extract is administered daily for at least about five weeks.

42. A method according to claim 25, wherein the extract is administered orally.

43. A method according to claim 31, wherein the extract is administered orally.

44. A method according to claim 42, wherein about 240 mg of said extract is administered.

45. A method according to claim 43, wherein about 240 mg of said extract is administered.

46. A method according to claim 44, wherein said extract is administered 1-10 times per day.

47. A method according to claim 45, wherein said extract is administered 1-10 times per day.

48. A method according to claim 44, wherein said extract is administered up to 4 times per day.

49. A method according to claim 45, wherein said extract is administered up to 4 times per day.

50. A method of improving cognitive ability in a human, comprising orally administering from 1-10 times per day to said human a composition comprising about 240 mg of an extract obtained from the bark of trees of the genus Pinus, wherein the human does not suffer from a neurodegenerative, neurological, psychiatric or behavioral disorder at the time of administration.

51. A method of improving cognitive ability in a human, comprising orally administering from 1-10 times per day to said human a composition comprising about 240 mg of one or more proanthocyanidins, wherein the human does not suffer from a neurodegenerative, neurological, psychiatric or behavioral disorder at the time of administration.

52. A method according to claim 51, wherein said one or more proanthocyanidins are obtained from the bark of trees of the genus Pinus.

53. A method according to claim 51, wherein said one or more proanthocyanidins are obtained from the bark of the Pinus radiata.

54. A method of preventing a decline in cognitive ability in a human, comprising administration of a composition to said human which comprises an extract obtained from the bark of trees of the genus Pinus in an amount effective to prevent a decline in cognitive abilities in said human, wherein the human does not suffer from any neurodegenerative, neurological, psychiatric or behavioral disorder at the time of administration.

55. A method according to claim 54, wherein the decline in cognitive ability is age-associated.

56. A method according to claim 54, wherein the cognitive ability is memory.

57. A method according to claim 54, wherein the cognitive ability is working memory.

58. A method according to claim 54, wherein the cognitive ability is the ability to concentrate.

59. A method according to claim 54, wherein the cognitive ability is cognitive processing speed.

60. A method according to claim 54, wherein one or more of learning ability, problem solving ability, comprehension, response times on cognitive tasks, and accuracy on cognitive tasks.

61. A method according to claim 54, wherein the extract is from the bark of the Pinus radiata.

62. A method according to claim 54, wherein the extract includes a mixture of flavonoid and non-flavonoid compounds.

63. A method according to claim 54, wherein the extract comprises proanthocyanidin compounds.

64. A method according to claim 63, wherein said proanthocyanidin compounds comprise dimers, trimers, oligomers and polymers of catechin and epicatechin.

65. A method according to claim 63, wherein said extract further comprises one or more other flavonoids and plant-phenolic compounds selected from the group consisting of catechin, epichatechin, gallocatechin, epigallocatechin, quercetin, dihydroquercetin, myricetin, astringenin, pinosylvin, taxifolin, stilbenes, hydroxylstilbenes, and phenolic acids.

66. A method according to claim 54, wherein the extract contains water soluble active compounds.

67. A method according to claim 54, wherein the extract is produced using a water-based extraction process.

68. A method according to claim 54, wherein the extract is administered daily.

69. A method according to claim 54, wherein the extract is administered daily for at least about five weeks.

70. A method according to claim 54, wherein the extract is administered orally.

71. A method according to claim 70, wherein about 240 mg of said extract is administered.

72. A method according to claim 71, wherein said extract is administered 1-10 times per day.

73. A method according to claim 71, wherein said extract is administered up to 4 times per day.

74. A method of preventing a decline in cognitive ability in a human, comprising orally administering from 1-10 times per day to said human a composition comprising about 240 mg of an extract obtained from the bark of trees of the genus Pinus, wherein the human does not suffer from a neurodegenarative, neurological, psychiatric or behavioral disorder at the time of administration.

75. A method of preventing a decline in cognitive ability in a human, comprising orally administering from 1-10 times per day to said human a composition comprising about 240 mg of one or more proanthocyanidins, wherein the human does not suffer from a neurodegenerative, neurological, psychiatric or behavioral disorder at the time of administration.

76. A method according to claim 75, wherein said one or more proanthocyanidins are obtained from the bark of trees of the genus Pinus.

77. A method according to claim 75, wherein said one or more proanthocyanidins are obtained from the bark of the Pinus radiata.

78. A method of improving cognitive processing speed in a human, comprising administering to said human a composition comprising an effective amount of proanthocyanidins wherein said human does not suffer from a neurodegenerative, neurological, psychiatric or behavioral disorder at the time of administration.

79. A method according to claim 78, wherein the composition of proanthocyanidins is derived from a plant extract.

80. A method according to claim 79, wherein the composition of proanthocyanidins is a bark extract from the bark of trees of the genus Pinus.

81. A method according to claim 80, wherein the bark extract is from the bark of the Pinus radiata.

82. A method according to claim 78, wherein the composition is administered orally.

83. A method according to claim 79, wherein the composition is administered orally.

84. A method according to claim 80, wherein the composition is administered orally.

85. A method according to claim 81, wherein the composition is administered orally.

86. A product comprising an extract obtained from the bark of trees of the genus Pinus and instructions for the administration of doses effective to improve cognitive ability in a human, wherein said human does not suffer from a neurodegenerative, neurological, psychiatric or behavioral disorder at the time of administration.

87. A product comprising an extract obtained from the bark of trees of the genus Pinus and instructions for the administration of doses effective to prevent a decline in cognitive ability in a human, wherein said human does not suffer from a neurodegenerative, neurological, psychiatric or behavioral disorder at the time of administration.

88. A product comprising an extract obtained from the bark of trees of the genus Pinus and instructions for the administration of doses effective to improve cognitive processing speed in a human, wherein said human does not suffer from a neurodegenerative, neurological, psychiatric or behavioral disorder at the time of administration.

89. A product comprising proanthocyanidins and instructions for the administration of doses effective to improve cognitive ability in a human, wherein said human does not suffer from a neurodegenerative, neurological, psychiatric or behavioral disorder at the time of administration.

90. A product comprising proanthocyanidins and instructions for the administration of doses effective to prevent a decline in cognitive ability in a human, wherein said human does not suffer from a neurodegenerative, neurological, psychiatric or behavioral disorder at the time of administration.

91. A product comprising proanthocyanidins and instructions for the administration of doses effective to improve cognitive processing speed in a human, wherein said human does not suffer from a neurodegenerative, neurological, psychiatric or behavioral disorder at the time of administration.

92. The product of any of claims 86-90 or 91, wherein said product comprises an extract from the bark of trees of the genus Pinus.

93. The product of any of claims 86-90 or 91, wherein said product comprises an extract from the bark of the Pinus radiata.

94. The product of claim 93, which further comprises one or more vitamins and/or minerals.

95. A food product suitable for human consumption, the improvement comprising the inclusion of proanthocyanidins in an amount effective to improve or prevent a decline in cognitive ability in a human.

96. A food product according to claim 95, wherein the food product is a solid food product.

97. A food product according to claim 95, wherein the food product is a liquid food product.

98. A food product according to any of claims 95, 96 or 97, wherein proanthocyanidins are provided in the form of an extract from the bark of trees of the genus Pinus.

99. A food product according to any of claims 95, 96 or 97, wherein proanthocyanidins are provided in the form of an extract from the bark of the Pinus radiata.
